(19) United States
(12) Patent Application Publication
(10) Pub. No.: US 2018/0064618 A1
(43) Pub. Date: Mar. 8, 2018

(54) WATER- OR ACID-TRIGGERED FRAGRANCE-RELEASE FUNCTIONAL MONOMER AND POLYMER SYSTEM

(71) Applicant: KIMBERLY-CLARK WORLDWIDE, INC., Neenah, WI (US)

(72) Inventors: Yiming Weng, Cumming, GA (US); Xuedong Song, Alpharetta, GA (US)

(21) Appl. No.: 15/557,932

(22) PCT Filed: Nov. 25, 2015
(86) PCT No.: PCT/US15/62711

§ 371 Date: Sep. 13, 2017

(51) Int. Cl. A61K 8/41; A61K 8/34; A61K 8/36; A61K 8/49; A61K 8/72; A61Q 13/00; A61Q 19/00

(52) U.S. Cl. CPC A61K 8/416; A61K 8/34; A61K 8/36; A61Q 19/00; A61K 8/72; A61Q 13/00; A61K 8/4973

(57) ABSTRACT

A triggerable composition for one-stage, controlled release of a functional chemical includes a functional monomer having a structure selected from the group described herein, wherein R is a polymerizable portion, N⁺X³¹ is a quaternary ammonium halide, and R′ and R″ are hydrocarbon-containing groups, wherein at least one of R′ and R″ includes a fragrance or a skin active chemical. At least one of R′ and R″ can be a ketone, R″ can be an aldehyde, and R′ can be an alcohol.

### BACKGROUND

The present disclosure pertains to a composition that controls the chemical release of functionally proactive components from a previously inactive and protected state. In particular, the present disclosure pertains to a composition that gradually or rapidly releases proactive chemical components upon the occurrence of specific environmental stimuli, where the composition can be used in bandages, hygiene products, health care products and skin-contacting beauty products, as well as in consumer product applications.

A large number of functionally proactive chemicals are known for use with personal care and beauty products, hygiene products, health-care related products, and skin-contacting products. For example, such proactives include antimicrobial or antibacterial agents, skin active chemicals including those with antioxidants, other antioxidant agents, antiseptic-type agents, skin repairing agents, and fragrances. Unfortunately, many of these functionally proactive chemicals are not stable under various environmental conditions. For example, if such proactives include volatile components, such as those found in fragrances, they can dissipate into the surrounding environment upon exposure to air and humidity conditions. Therefore such chemicals can demonstrate short shelf lives when in use, and present serious packaging/storage concerns. As a result, costly packaging requirements can be necessary for products incorporating such chemicals. This instability therefore creates a significant limitation on the wide adoption of the potentially useful chemistry, and limits the long-term efficacy of products incorporating such chemistry. Further, processing challenges such as elevated temperatures can exist, and, as a result, can present a need to limit exposure to environmental stimuli during manufacture.

Additional challenges include the difficulties involved with controlling the gradual release of such proactive chemicals, as well as the potential side effects/costs resulting from use of chemically-degraded products. Other proactives, such as antioxidants, are also often not stable when exposed to ambient conditions, such as the air of a user's pantry or storage closets. Antioxidants can readily be oxidized by oxygen in the air. A need therefore exists for a versatile composition that effectively stabilizes functional chemical proactives, and releases such proactives upon demand, at a desirable rate and profile.

Certainly, attempts have been made to overcome the stability and storage limitations presented by such proactives. For example, attempts have been suggested for stabilizing retinol by encapsulating it in pH-sensitive polymers and then releasing it at a later time by changing the solubility of the encapsulating matrix through a pH change. The encapsulated retinol still suffers significant degradation, presumably from oxidation. Others have suggested overcoming such stability issues by converting retinol into an ester as a proactive (a precursor to the retinol active) and then at a later time converting the ester into the proactive form by use of enzymes present in a user's body after delivery through a user's skin. In this approach, however, only a small portion of the ester is used effectively by the skin layer and a majority of the esters are wasted by the system. Such a system can also actually lead to side effects when too much retinol ester is used to achieve effective dosages on the skin. Therefore, a need still exists for delivery compositions for skin repair proactives.

In connection with the delivery of fragrances (such as in connection with personal care absorbent products), it has been suggested to encapsulate fragrances in polymeric matrices for stabilization and delivery benefits. However, even with such encapsulation technology, there is a further need for fragrance encapsulation technology that offers effective protection for such volatiles as well as a controlled release. Existing encapsulation chemistries for consumer products often leak or release prematurely. Therefore a continuing need exists for a material composition that provides both stability for unstable proactives and the release of proactives in a controlled manner.

### SUMMARY

The present disclosure describes a polymeric proactive system for fragrance delivery. Polymeric proactive systems for drug delivery have been studied and applied for years. A novel water-triggered polymeric proactive system for fragrance delivery is described. Polymerizable fragrances with a water-trigger function were synthesized and characterized.

The present disclosure describes a triggerable composition for creating a stable, controlled-release of functional chemical proactive components, using a one-stage release mechanism. The graduated or rapid release of functional chemical proactive components allows for protection of the functional proactives from the surrounding environment, as well as the selective release of such proactives, upon the occurrence of a select environmental stimulus. For the purposes of this application, the term “aqueous medium” shall mean a medium containing “liquid” water as opposed to water vapor. Examples of an aqueous medium include urine, sweat, vaginal fluids, mucous, menses, and runny, liquid, and loose bowel movements.

In one aspect of the disclosure, a triggerable composition for one-stage, controlled release of a functional chemical includes a functional monomer having a structure selected from the group consisting of

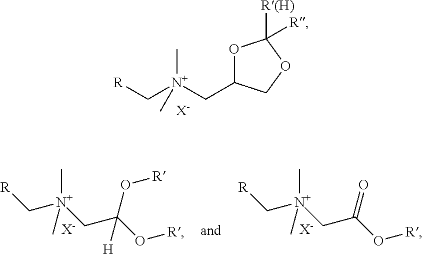

wherein R is a polymerizable portion, N⁺X⁻ is a quaternary ammonium halide, and R′ and R″ are hydrocarbon-containing groups, wherein at least one of R′ and R″ includes a fragrance.

In an alternative aspect of the disclosure, a coating includes a triggerable composition for one-stage, controlled release of a functional chemical, the composition including a functional monomer having a structure selected from the group consisting of

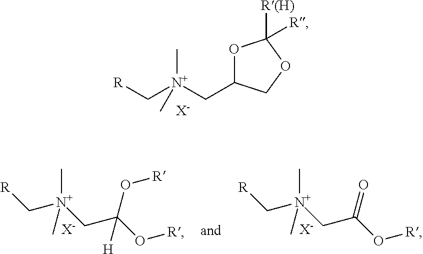

wherein R is a polymerizable portion, N+X− is a quaternary ammonium halide, and R′ and R″ are hydrocarbon-containing groups, wherein at least one of R′ and R″ includes a fragrance.

In yet another alternative aspect of the disclosure, a skin care element includes a triggerable composition for one-stage, controlled release of a functional chemical, the composition including a functional monomer having a structure selected from the group consisting of

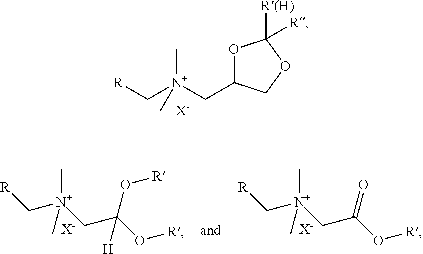

wherein R is a polymerizable portion, N⁺X⁻ is a quaternary ammonium halide, and R′ and R″ are hydrocarbon-containing groups, wherein at least one of R′ and R″ includes a skin active chemical.

Other features and aspects of the present disclosure are discussed in greater detail below.

### BRIEF DESCRIPTION OF FIGURES

The present disclosure will be more fully understood, and further features will become apparent, when reference is made to the following detailed description and the accompanying drawings. The drawings are merely representative and are not intended to limit the scope of the claims.

FIG. 1 illustrates the scheme of water-or acid-sensitive fragrance synthesis and hydrolysis;

FIG. 2 illustrates the preparation of water-or acid/base-triggered fragrance-release polymers containing an aldehyde or ketone as a releasable fragrance;

FIG. 3 illustrates an example of the synthesis of a cyclic acetal monomer and its constructed polymer;

FIG. 4 illustrates the preparation of water-or acid/base-triggered fragrance-release polymers containing an alcohol as a releasable fragrance;

FIG. 5 illustrates the synthesis of monomers: (1) synthesis of fragrance monomers with ester-linkage; (2) synthesis of fragrance monomers imine-linkage; (3) synthesis of a reactive allylamine monomer;

FIG. 6 illustrates an example of the synthesis of an ester-linked monomer and its constructed polymer;

FIG. 7 is a graphical representation of FT-IR spectra (from bottom to top): (1) ATP; (2) CPD; (3) AC; (4) DMAPMAm; (5) DAC;

FIG. 8 is a graphical representation of FT-IR spectra (from bottom to top): (1) DAC; (2) PVP; (3) P(NVP-co-DAC);

FIG. 9 is a graphical representation of FT-IR spectra (from bottom to top): (1) DAC; (2) PMMA; (3) P(MMA-co-DAC);

FIG. 10 is a graphical representation of FT-IR spectra (from bottom to top): (1) DHC; (2) PVP; (3) P(NVP-co-DHC);

FIG. 11 is a graphical representation of FT-IR spectra (from bottom to top): (1) DHC; (2) PMMA; (3) P(MMA-co-DHC);

FIG. 12 is a graphical representation of FT-IR spectra (from bottom to top): (1) EH; (2) ECA; (3) DMAPMAm; (4) DECA;

FIG. 13 is a graphical representation of FT-IR spectra (from bottom to top): (1) ECA; (2) EBA;

FIG. 14 is a graphical representation of FT-IR spectra (from bottom to top): (1) PNVPDMA; (2) PNVPDMA-g-CPD; (3) PNVPDMA-g-CPDAld; and (4) PNVPDMA-g-CPD-AldEH;

FIG. 15 is a graphical representation of FT-IR spectra (from bottom to top): (1) PNVPDMA; (2) PNVPDMA-g-CPD; (3) PNVPDMA-g-CPDHAL; and

FIG. 16 is a graphical representation of FT-IR spectra (from bottom to top): (1) PNVPDMA-g-CPDHAL; (2) PNVPDMA-g-CPDCAL; (3) PNVPDMA-g-CPDATP.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

This present disclosure describes a polymeric proactive system for fragrance delivery. The polymers must be biocompatible (non-toxic), capable of loading the proactive, and able to release the proactive via a trigger mechanism. This system includes the synthesis of functional monomers containing quaternary ammonium salts as an unstable trigger for both ester-and cyclic acetal-linkages in the presence of water, and then attaching the functional monomers onto polymers for a quick release of the fragrance. The monomers are either polymerized to make related homopolymers or co-polymerize with other monomers to make copolymers for controlled release of the proactives.

Examples are demonstrated using polymers containing an aldehyde, ketone, or alcohol as a releasable fragrance. One benefit of these polymeric proactive systems is that the polymer is able to protect the proactive in high humidity situations and is much more stable. Potential applications include fragrance release, wetness indications, and skin care/actives delivery. Examples of proactives that can be used in the present application include antimicrobial or antibacterial agents, skin active chemicals including those with antioxidants, other antioxidant agents, antiseptic-type agents, skin repairing agents, and fragrances.

Synthesis of monomers with an aldehyde or a ketone as a releasable fragrance involves the synthesis of a cyclic acetal fragrance intermediate with halogen at the end, followed by either converting the cyclic acetal fragrance intermediate with halogen at the end to a polymerizable monomer or attaching the cyclic acetal fragrance intermediate with halogen at the end to a pre-formed polymer. An alternative approach is to attach 3-halo-1,2-propanediol to the pre-formed polymer and then form a cyclic acetal with an aldehyde/ketone-based fragrance.

Synthesis of monomers with an alcohol as a releasable fragrance via an acyclic acetal linkage involves the polymer synthesis, attachment of quaternary ammonium salt intermediate, conversion of 1,2-diol to an aldehyde, and finally formation of a polymer with a pendent acetal-based fragrance.

These compositions allow for a single-stage triggered release of a fragrance.

The surrounding environment or targeted location can be onto a user's skin, or into the structure of an article containing the triggerable composition. Such article can be for example, a health care product, such as a garment or bandage, a hygiene product such as a tissue or wipe, a skin-contacting beauty product such as a facial wrap, an absorbent consumer/personal care article, such as a feminine care pad or liner, a baby or child care diaper, or an adult incontinence garment. The composition of the disclosure can further be present in a lotion, cream or medicament as well.

Polymers as a delivery vehicle for proactives have been popular in pharmaceutical, biomedical, and consumer product applications. The most common examples include the use of polymers to deliver drugs, cells, proteins, and genes. For the present disclosure, polymers were used to carry and deliver fragrances for a variety of consumer products including feminine care products, diapers, and paper-related products.

Because the polymers developed in this disclosure are used for human consumer products, the polymers must be biocompatible and cannot have any potential toxicity. Also, the polymers must be designed to have the capability to load the proactives. The polymers must be able to release the proactives in the surrounding environment spontaneously or by a trigger. Lastly, the polymers must be designed to be capable of attaching to or incorporating into consumer products.

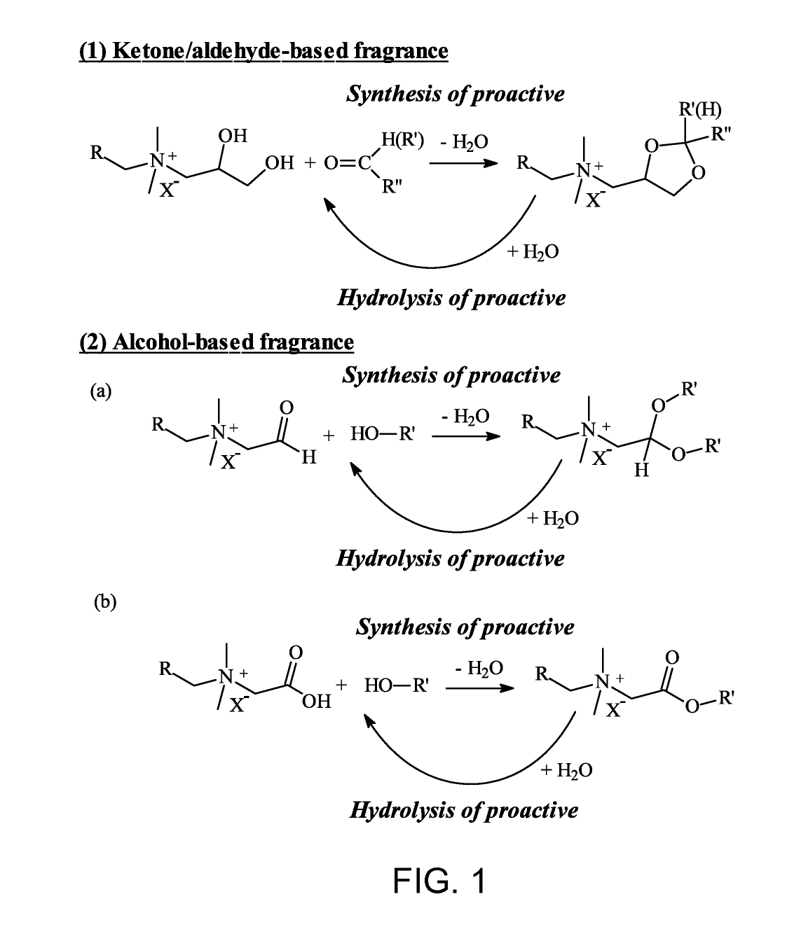

To meet these requirements, functional monomers containing quaternary ammonium salts as an unstable trigger for both ester-and cyclic acetal-linkages in the presence of water were synthesized and then attached onto polymers for a quick release of the fragrance, as shown in FIG. 1. Both alcohol and aldehyde/ketone-containing functional fragrance monomers were synthesized for this project. These synthesized fragrance-releasable polymers can be either directly attached to the surface of a product or substrate via physical and/or chemical interaction or delivered in the form of mixing or insertion of crosslinked gels or particles.

### Preparation of Water-or Acid-Triggered Fragrance-Release Polymers

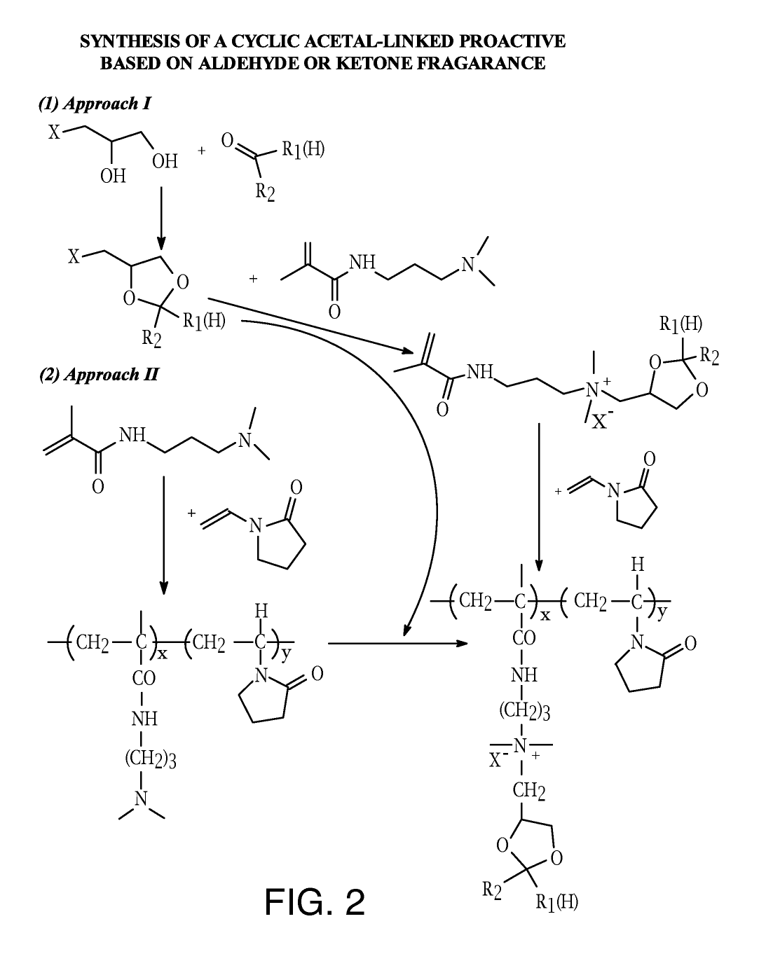

1) A) Synthesis of polymers containing an aldehyde or a ketone as a releasable fragrance: Synthesis of monomers with an aldehyde or a ketone as a releasable fragrance used a procedure described below (see FIG. 2). Essentially, preparation involved the synthesis of a cyclic acetal fragrance intermediate with halogen at the end, followed by either converting it to a polymerizable monomer via Approach l or attaching it to a pre-formed polymer via Approach II, both shown in FIG. 2. In another alternative (not shown), Approach III, 3-halo-1,2-propanediol was attached to the pre-formed polymer and then a cyclic acetal with an aldehyde/ketone-based fragrance was formed, step by step. For synthesis of a cyclic acetal intermediate to a ketone-or aldehyde-containing fragrance in toluene in the presence of sulfuric acid (catalytic amount), 3-chloro-1,2-propanediol was added. The reaction was refluxed at 110-120° C. for 4-5 hours, followed by washing with sodium bicarbonate solution and saturated sodium chloride solution. After drying with anhydrous magnesium sulfate, the toluene was completely removed with a rotary evaporator.

For attaching the cyclic intermediate onto polymers, two approaches were applied. In Approach I, the purified cyclic acetal fragrance was used to react with N-(3-dimethylamino)propyl methacrylamide (DMAPMAm) at 70-80° C. for 3-5 hours to form a quaternary ammonium derivative with polymerizable C═C at the end, i.e., the new fragrance molecule bearing acetal, quaternary ammonium halide and carbon-carbon double bond functional groups. Then this functional monomer was directly used to copolymerize with a variety of monomers such as N-vinylpyrrolidone (NVP), 2-hydroxyethyl methacrylate (HEMA), methyl methacrylate (MMA), etc., in the presence of Azobisisobutyronitrile (AIBN) at 65-70° C. under N₂ for 3-5 hours, to form a polymer capable of releasing fragrance in the presence of water or acidic aqueous solution.

In Approach II, a polymer containing tertiary amine functionalities was synthesized. Briefly, N-(3-dimethylaminopropyl)methacrylamide was copolymerized with a variety of monomers such as N-vinylpyrrolidone (NVP), 2-hydroxyethyl methacrylate (HEMA), methyl methacrylate (MMA), etc., in the presence of AIBN at 65-70° C. under N₂ for 3-5 hours, to form the polymer containing tertiary amine functionalities. Then the cyclic acetal fragrance intermediate in toluene or ethanol was attached to the polymer at 70-80° C. for 3-5 hours, followed by precipitation with ether or direct use without further purification.

In Approach III, a polymer containing tertiary amine functionalities was first synthesized as shown above in Approach II. Then 3-halo-1,2-propanediol was attached to the polymer at 70-80° C. for 5-6 hours via a quaternary ammonium halide formation. Finally, aldehyde or ketone-based fragrance was attached based on an acetal formation at room temperature in the presence of Lewis acid, p-toluenesulfonic acid and molecular sieves, followed by precipitation and washing with ether.

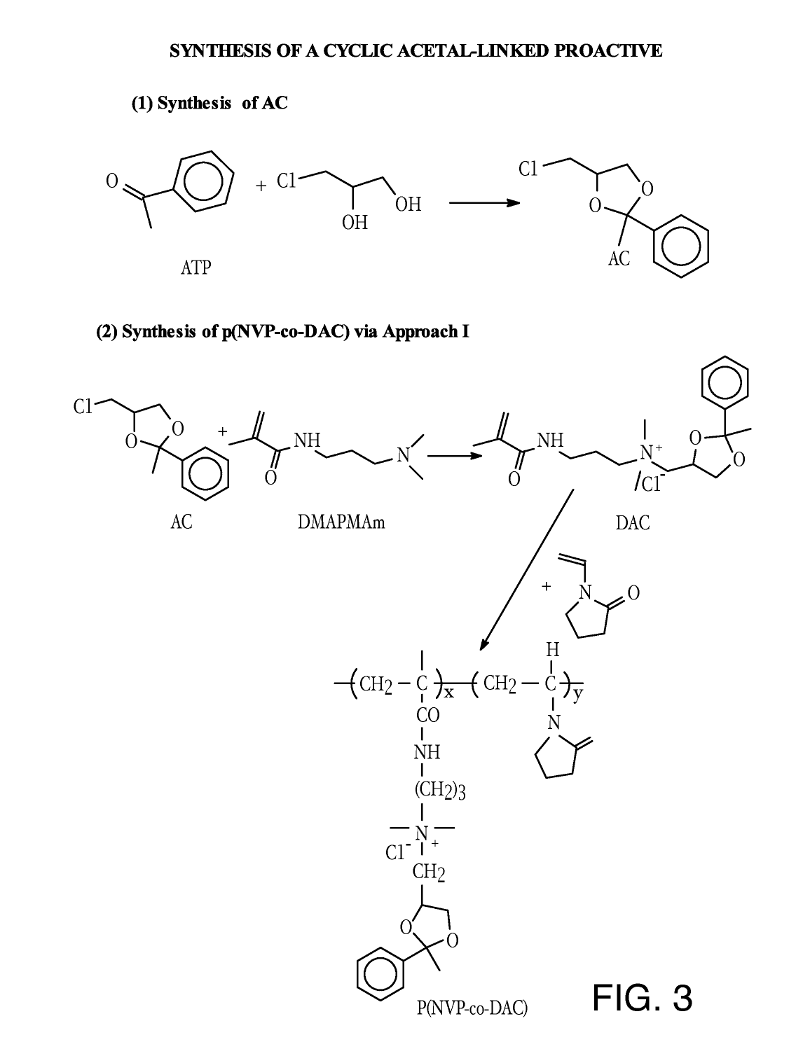

1) B) Examples of synthesis of polymers containing an aldehyde or a ketone as a releasable fragrance: A typical synthesis example is shown in FIG. 3 and described below: a ketone-type fragrance compound acetophenone (ATP) was used to react with ±3-chloro-1,2-propanediol (CPD) to form a cyclic acetal ATP-CPD (AC). With Approach I, AC was used to react with N-[3-(Dimethylamino)propyl] methacrylamide (DMAPMAm) to form DAC (see FIG. 3). The DAC was copolymerized with N-vinylpyrrolidone (NVP) to form a poly(NVP-co-DAC) copolymer. With Approach II, AC was directly attached to the polymer containing tertiary amine groups, i.e., poly(NVP-co-DMAPMAm), to form the poly(NVP-co-DAC) copolymer.

Another example employs the synthesis of a polymer containing an aldehyde-based fragrance. The fragrance heptanal (HNL) was used to react with CPD to form a cyclic acetal HNL-CPD (HC). With Approach I, HC was used to react with DMAPMAm to form DHC, followed by forming a copolymer P(NVP-co-DHC). Likewise, by copolymerization with hydrophobic monomer MMA, DHC can form a poly(MMA-co-DHC) copolymer.

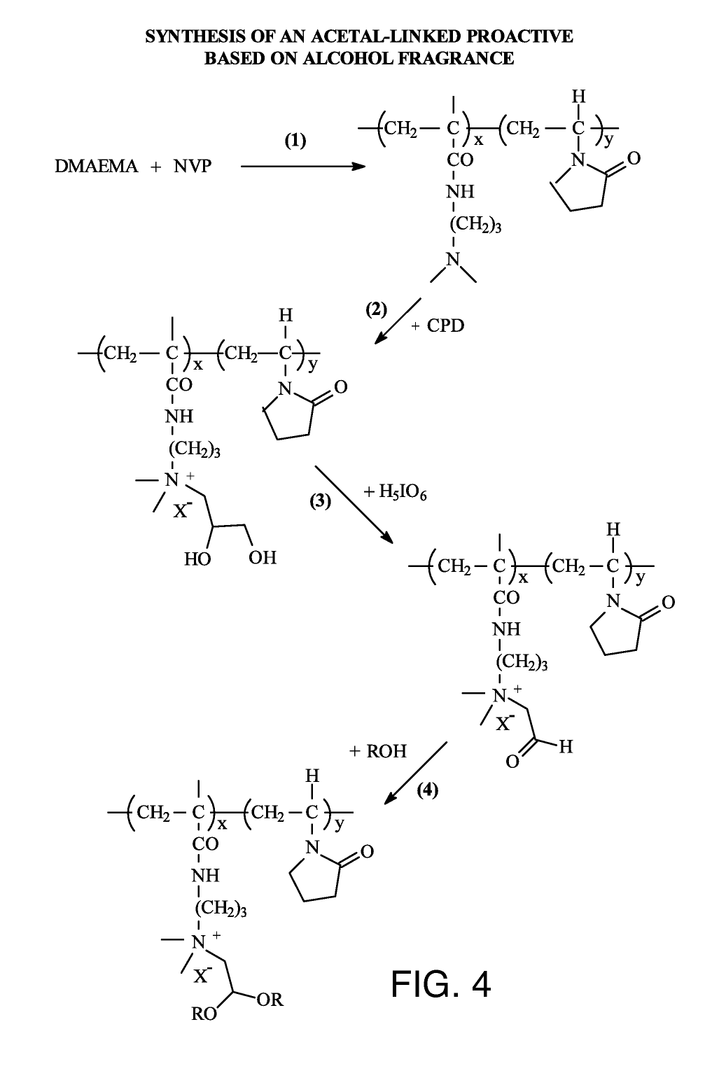

2) A) Synthesis of polymers containing an alcohol as a releasable fragrance via an acyclic acetal linkage: Synthesis of monomers with an alcohol as a releasable fragrance via an acyclic acetal linkage was conducted using a procedure described below (see FIG. 4). Essentially, preparation involved the polymer synthesis, attachment of quaternary ammonium salt intermediate, conversion of 1,2-diol to an aldehyde, and finally formation of a polymer with a pendent acetal-based fragrance. Briefly, polymers containing tertiary amine groups were synthesized in DMAc in the presence of AIBN initiator at 65-70° C. under N₂ blanket for 4-5 hours. The formed polymers were directly used for the next step without purification. CPD was added to the polymer solution. The mixture was heated to 70-80° C. and kept at that temperature for 6-7 hours, followed by precipitating with ether. After the precipitated polymers were dissolved in distilled water, periodic acid was added. The reaction was run at room temperature for 3-4 hours, followed by purification with membrane dialysis against distilled water and drying with freeze-drying. After freeze-drying, the purified polymers were dissolved in DMAc again. The alcohol-based fragrance was added to the solution. The reaction was run in the presence of p-toluenesulfonic acid at room temperature for 8-9 hours, followed by precipitation with ether. After washing with ether several times, the purified polymers were dried and stored in vacuo.

2) B) Example of synthesis of polymers containing an alcohol as a releasable fragrance via an acyclic acetal linkage: In a typical synthesis example, NVP was used to copolymerize with DMAEMA in the presence of AIBN under N₂ blanket at 70° C. for 8 hours, followed by adding CPD at 75-80° C. for 6 hours. The copolymer was precipitated with ether and dissolved in distilled water. With the addition of periodic acid, the aldehyde-containing polymer was formed after the reaction was run at room temperature for 4 hours. The polymer was then dialyzed against distilled water overnight, followed by freeze-drying. The dried polymer was finally used to react with the fragrance compound, 2-ethyl-1-hexanol (EH), in the presence of p-toluenesulfonic acid at room temperature overnight. After precipitation and washing with ether, the polymer was dried and stored in a vacuum oven.

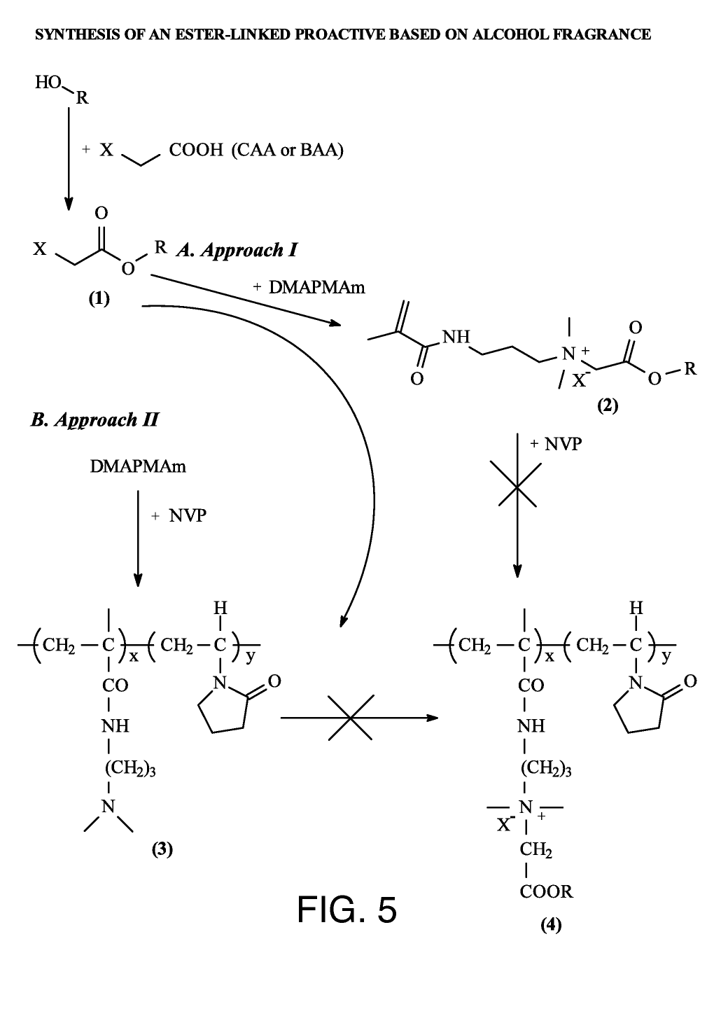

3) A) Synthesis of polymers containing an alcohol as a releasable fragrance via an ester-linkage: Synthesis of monomers with an alcohol as a releasable fragrance via an ester-linkage was conducted using a procedure described below (see FIG. 5). Essentially, preparation involves the synthesis of an ester-linked fragrance intermediate with halogen at the end, followed by either converting it to a polymerizable monomer via Approach I or attaching it to a pre-formed polymer via Approach II, as shown in FIG. 5. For synthesis of an ester-linked fragrance intermediate, chloroacetic acid or bromoacetic acid was added to an alcohol-containing fragrance in toluene in the presence of sulfuric acid (catalytic amount). The reaction was refluxed at 110-120° C. for 4-5 hours, followed by washing with sodium bicarbonate solution and saturated sodium chloride solution. After drying with anhydrous magnesium sulfate, the toluene was completely removed with a rotary evaporator.

For attaching the ester-linked fragrance intermediate onto polymers, two approaches were applied. In Approach I, the purified fragrance intermediate was used to react with N-(3-dimethylamino)propyl methacrylamide (DMAPMAm) at 70-80° C. for 3-5 hours to form a quaternary ammonium derivative with polymerizable C═C at the end, i.e., the new fragrance molecule bearing ester, quaternary ammonium and carbon-carbon double bond functional groups. Then this functional monomer was directly used to copolymerize with a variety of monomers such as N-vinylpyrrolidone (NVP), 2-hydroxyethyl methacrylate (HEMA), methyl methacrylate (MMA), etc., in the presence of AIBN at 65-70° C. under N₂ for 3-5 hours, to form a polymer capable of releasing fragrance in the presence of water.

In Approach II, a polymer containing tertiary amine functionalities was synthesized. Briefly, DMAPMAm was copolymerized with a variety of monomers such as N-vinylpyrrolidone (NVP), 2-hydroxyethyl methacrylate (HEMA), methyl methacrylate (MMA), etc., in the presence of AIBN at 65-70° C. under N₂ for 3-5 hours, to form the polymer containing tertiary amine functionalities. Then the ester-linked fragrance intermediate in toluene or ethanol was attached onto the polymer at 70-80° C. for 3-5 hours, followed by precipitation with ether or direct use without further purification.

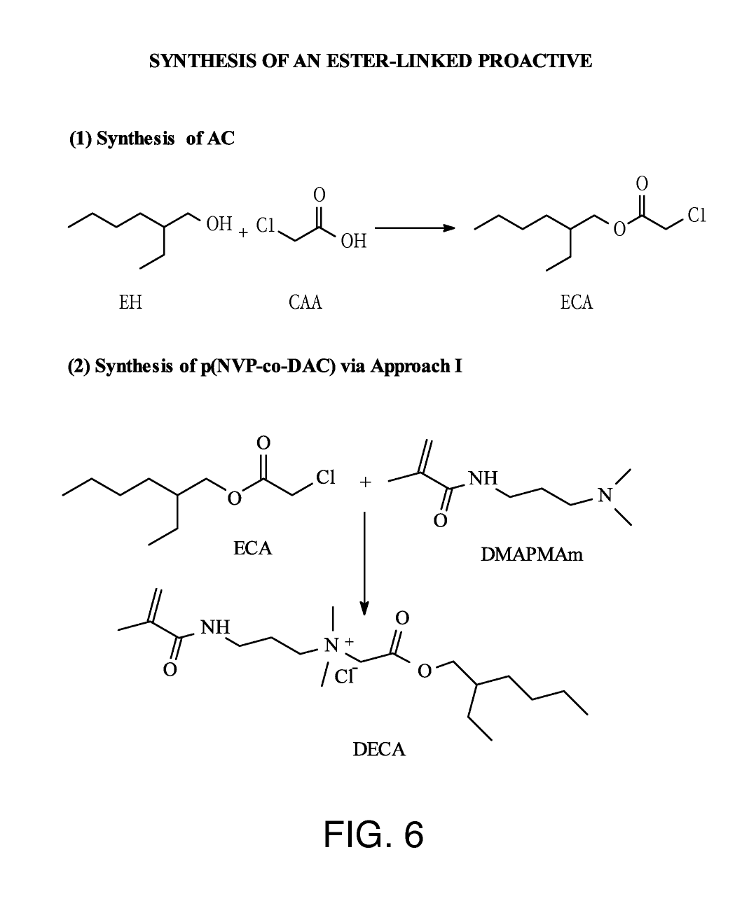

3) B) Example of synthesis of polymers containing an alcohol as a releasable fragrance: In a typical synthesis example, fragrance compound 2-ethyl-1-hexanol (EH) was used to react with chloroacetic acid (CAA) to form an ester-linked and chlorine-containing fragrance (ECA). Then with Approach I, ECA was used to react with DMAPMAm to form DECA (see FIG. 6). Bromoacetic acid (BAA) was also used to form EBA with EH, followed by reacting with DMAPMAm to form DEBA.

### EXAMPLES

### Examples 1-39

### Monomer and Polymer Synthesis

### Example 1

Synthesis of AC: 3-chloro-1,2-propanediol (7 parts, CPD) was added to acetophenone (9.7 parts, ATP) in toluene (35 parts) in the presence of sulfuric acid (1.5 parts). The reaction was refluxed at 110-120° C. for 4-5 hours, followed by washing with sodium bicarbonate solution and saturated sodium chloride solution. After drying with anhydrous magnesium sulfate and completely removing toluene via a rotary evaporator, the product AC (ATP-CPD) was obtained.

### Example 2

Synthesis of HC: CPD (7.3 parts) was added to heptanal (12.2 parts, HNL) in toluene (35 parts) in the presence of sulfuric acid (1.5 parts). The reaction was refluxed at 110-120° C. for 4-5 hours, followed by washing with sodium bicarbonate solution and saturated sodium chloride solution. After drying with anhydrous magnesium sulfate and completely removing toluene via a rotary evaporator, the product HC (HNL-CPD) was obtained.

### Example 3

Synthesis of DAC: N-(3-dimethylamino)propyl methacrylamide (4 parts, DMAPMAm) was added to AC (5 parts) in toluene (25 parts). After the reaction was run at 70-80° C. for 3-5 hours, the product was left in the solution for the next reaction.

### Example 4

Synthesis of DHC: DMAPMAm (8.2 parts) was added to HC (10 parts) in toluene (50 parts). After the reaction was run at 70-80° C. for 3-5 hours, the product was left in the solution for the next reaction.

### Example 5

Synthesis of the NVP-containing copolymer in toluene: N-vinylpyrrolidone (60 parts, NVP) and azobisisobutyronitrile (2.3 parts, AIBN) were added to DAC (90 parts) in toluene (500 parts). The reaction was run at 65-70° C. under N₂ for 3-5 hours, followed by precipitation with ether to produce solid polymer powders.

### Example 6

Synthesis of the NVP-containing copolymer in ethanol: NVP (20 parts) and AIBN (0.7 parts) were added to DAC (30 parts) in ethanol (150 parts). After the reaction was run at 65-70° C. under N₂ for 5-6 hours, the reaction was stopped and the polymer product was left in ethanol without further purification for direct use.

### Example 7

Synthesis of the NVP-containing copolymer in methanol/ethanol: NVP (10 parts) and AIBN (0.4 parts) were added to DAC (15 parts) in methanol (35 parts) and ethanol (35 parts). The reaction was run at 65-70° C. under N₂ for 3-5 hours, followed by precipitation with ether to produce solid polymer powders.

### Example 8

Synthesis of the NVP-containing copolymer in toluene: NVP (61 parts) and AIBN (2.3 parts) were added to DHC (89 parts) containing toluene (450 parts). The reaction was run at 65-70° C. under N₂ for 3-5 hours, followed by precipitation with ether to produce solid polymer powders.

### Example 9

Synthesis of the NVP-containing copolymer in ethanol: NVP (20 parts) and AIBN (0.7 parts) were added to DHC (30 parts) in ethanol (250 parts). After the reaction was run at 65-70° C. under N₂ for 5-6 hours, the reaction was stopped and the polymer product was left in ethanol without further purification for direct use.

### Example 10

Synthesis of the MMA-containing copolymer: Methyl methacrylate (57 parts, MMA) and AIBN (1.5 parts) were added to DAC (93 parts) containing toluene (400 parts). The reaction was run at 65-70° C. under N₂ for 3-5 hours, followed by precipitation with ether to produce solid polymer powders.

### Example 11

Synthesis of the MMA-containing copolymer in ethanol: MMA (19 parts) and AIBN (0.5 parts) were added to DAC (31 parts) in ethanol (130 parts). After the reaction was run at 65-70° C. under N₂ for 5-6 hours, the reaction was stopped and the polymer product was left in ethanol without further purification for direct use.

### Example 12

Synthesis of the HEA-containing copolymer: 2-hydroxyethyl acrylate (31 parts, HEA) and AIBN (0.8 parts) were added to DAC (44 parts) containing toluene (250 parts). The reaction was run at 65-70° C. under N₂ for 3-5 hours, followed by precipitation with ether to produce solid polymer powders.

### Example 13

Synthesis of poly(NVP-co-DMAPMAm) copolymer: NVP (90 parts) and DMAPMAm (60 parts) were added to AIBN (1.5 parts) containing toluene (450 parts). After a 30-minute nitrogen purging, the reaction was run at 65-70° C. for 3-5 hours. Then the solution was directly used for halogen-containing fragrance tethering.

### Example 14

Synthesis of poly(MMA-co-DMAPMAm) copolymer: MMA (44 parts) and DMAPMAm (32 parts) were added to AIBN (0.7 parts) containing toluene (250 parts). After a 30-minute nitrogen purging, the reaction was run at 65-70° C. for 3-5 hours. Then the solution was directly used for halogen-containing fragrance tethering.

### Example 15

Synthesis of poly(NVP-co-DMAEMA) copolymer: NVP (47 parts) and 2-(dimethylamino)ethyl methacrylate (29 parts, DMAEMA) were added to AIBN (0.7 parts) containing toluene (250 parts). After a 30-minute nitrogen purging, the reaction was run at 65-70° C. for 3-5 hours. Then the solution was directly used for halogen-containing fragrance tethering.

### Example 16

Synthesis of poly(NVP-co-DMAEA) copolymer: NVP (66 parts) and 2-(dimethylamino)ethyl acrylate (84 parts, DMAEA) were added to AIBN (1.5 parts) containing toluene (450 parts). After a 30-minute nitrogen purging, the reaction was run at 65-70° C. for 3-5 hours. Then the solution was directly used for halogen-containing fragrance tethering.

### Example 17

Synthesis of AC-containing poly(NVP-co-DMAPMAm) copolymer: AC (4 parts) from Example 1 was added to poly(NVP-co-DMAPMAm) copolymer (10 parts) from Example 13 in toluene (35 parts). The reaction was run at 70-80° C. for 3-5 hours, followed by precipitation with ether to produce solid polymer powders.

### Example 18

Synthesis of HC-containing poly(NVP-co-DMAPMAm) copolymer: HC (7.7 parts) from Example 2 was added to poly(NVP-co-DMAPMAm) copolymer (20 parts) from Example 13 in toluene (60 parts). The reaction was run at 70-80° C. for 3-5 hours, followed by precipitation with ether to produce solid polymer powders.

### Example 19

Synthesis of AC-containing poly(MMA-co-DMAPMAm) copolymer: AC (4.2 parts) from Example 1 was added to poly(MMA-co-DMAPMAm) copolymer (10 parts) from Example 14 in toluene (30 parts). The reaction was run at 70-80° C. for 3-5 hours, followed by precipitation with ether to produce solid polymer powders.

### Example 20

Synthesis of HC-containing poly(MMA-co-DMAPMAm) copolymer: HC (5.5 parts) from Example 2 was added to poly(MMA-co-DMAPMAm) copolymer (15 parts) from Example 14 in toluene (40 parts). The reaction was run at 70-80° C. for 3-5 hours, followed by precipitation with ether to produce solid polymer powders.

### Example 21

Synthesis of ECA: Chloroacetic acid (7.3 parts, CAA) was added to 2-ethyl-1-hexanol (10 parts, EH) in toluene (50 parts) in the presence of sulfuric acid (0.5 parts). The reaction was refluxed at 110-120° C. for 4-5 hours, followed by washing with sodium bicarbonate solution and saturated sodium chloride solution. After drying with anhydrous magnesium sulfate and completely removing toluene via a rotary evaporator, the product ECA (EH-CAA) was obtained.

### Example 22

Synthesis of EBA: Bromoacetic acid (11 parts, BAA) was added to EH (20 parts) in toluene (100 parts) in the presence of sulfuric acid (1 part). The reaction was refluxed at 110-120° C. for 4-5 hours, followed by washing with sodium bicarbonate solution and saturated sodium chloride solution. After drying with anhydrous magnesium sulfate and completely removing toluene via a rotary evaporator, the product EBA (EH-BAA) was obtained.

### Example 23

Synthesis of CNCA: CAA (9 parts) was added to citronellol (15 parts, CN) in toluene (70 parts) in the presence of sulfuric acid (0.8 parts). The reaction was refluxed at 110-120° C. for 4-5 hours, followed by washing with sodium bicarbonate solution and saturated sodium chloride solution. After drying with anhydrous magnesium sulfate and completely removing toluene via a rotary evaporator, the product CNCA (CN-CAA) was obtained.

### Example 24

Synthesis of CNBA: BAA (8.9 parts) was added to CN (10 parts) in toluene (50 parts) in the presence of sulfuric acid (0.5 parts). The reaction was refluxed at 110-120° C. for 4-5 hours, followed by washing with sodium bicarbonate solution and saturated sodium chloride solution. After drying with anhydrous magnesium sulfate and completely removing toluene via a rotary evaporator, the product CNBA (CN-BAA) was obtained.

### Example 25

Synthesis of DECA: DMAPMAm (4.1 parts) was added to ECA (5 parts) in toluene (30 parts). After the reaction was run at 70-80° C. for 3-5 hours, the product was left in the solution for the next reaction.

### Example 26

Synthesis of DEBA: DMAPMAm (6.8 parts) was added to EBA (10 parts) in toluene (60 parts). After the reaction was run at 70-80° C. for 3-5 hours, the product was left in the solution for the next reaction.

### Example 27

Synthesis of ECA-containing poly(NVP-co-DMAPMAm) copolymer: ECA (5.5 parts) from Example 21 was added to poly(NVP-co-DMAPMAm) copolymer (15 parts) from Example 13 in toluene (45 parts). After the reaction was run at 70-80° C. for 10 min, the polymers entirely became gels.

### Example 28

Synthesis of ECA-containing poly(MMA-co-DMAPMAm) copolymer: ECA (5 parts) from Example 21 was added to poly(MMA-co-DMAPMAm) copolymer (12 parts) from Example 14 in toluene (36 parts). After the reaction was run at 70-80° C. for 10 min, the polymers entirely became gels.

### Example 29

Synthesis of EBA-containing poly(NVP-co-DMAPMAm) copolymer: EBA (4.7 parts) from Example 22 was added to poly(NVP-co-DMAPMAm) copolymer (10 parts) from Example 13 in toluene (30 parts). After the reaction was run at room temperature for 5 min, the polymers entirely became gels.

### Example 30

Synthesis of CNCA-containing poly(NVP-co-DMAPMAm) copolymer: CNCA (8 parts) from Example 23 was added to poly(NVP-co-DMAPMAm) copolymer (15 parts) from Example 13 in toluene (45 parts). After the reaction was run at 70-80° C. for 10 min, the polymers entirely became gels.

### Example 31

Synthesis of CNCA-containing poly(MMA-co-DMAPMAm) copolymer: CNCA (2.8 parts) from Example 23 was added to poly(MMA-co-DMAPMAm) copolymer (5 parts) from Example 14 in toluene (15 parts). After the reaction was run at 70-80° C. for 10 min, the polymers entirely became gels.

### Example 32

Synthesis of CNBA-containing poly(NVP-co-DMAPMAm) copolymer: CNBA (9.7 parts) from Example 24 was added to poly(NVP-co-DMAPMAm) copolymer (15 parts) from Example 13 in toluene (45 parts). After the reaction was run at room temperature for 5 min, the polymers entirely became gels.

### Example 33

Synthesis of the NVP-containing copolymer: NVP (2 parts) and AIBN (0.1 part) were added to DECA (3 parts) containing toluene (15 parts). After the reaction was run at 70-80° C. for 10 min, the polymers entirely became gels.

### Example 34

Synthesis of the MMA-containing copolymer: MMA (7.7 parts) and AIBN (0.2 parts) were added to DECA (7.3 parts) containing toluene (45, parts). After the reaction was run at 70-80° C. for 10 min, the polymers entirely became gels.

### Example 35

Synthesis of the NVP-containing copolymer: NVP (5.7 parts) and AIBN (0.15 parts) were added to DEBA (9.3 parts) containing toluene (45 parts). After the reaction was run at room temperature for 5 min, the polymers entirely became gels.

### Example 36

Synthesis of EHC-containing poly(NVP-co-DMAEMA) copolymer: CPD (1.3 parts) was added to poly(NVP-co-DMAEMA) copolymer or PVPDM (5 parts) from Example 15 in dimethylformamide (15 parts). After the reaction was run at 75-80° C. for 6 hours, the copolymer (PVPDM-g-CPD) was precipitated with ether and dissolved in distilled water. With addition of periodic acid (3 parts), the aldehyde-containing polymer (PVPDM-g-CPDAId) was formed after the reaction was run at room temperature for 4 hours. The polymer was then dialyzed against distilled water overnight, followed by freeze-drying. The dried polymer in dimethylformamide (10 parts) was directly used to react with the fragrance compound, 2-ethyl-1-hexanol (EH, 4.5 parts), in the presence of p-toluenesulfonic acid (0.9 part) at room temperature overnight. After precipitation and washing with ether, the polymer (PVPDM-g-CPDAIdEH) was dried and stored in a vacuum oven.

### Example 37

Synthesis of HALO-containing poly(NVP-co-DMAEMA) copolymer: CPD (1.3 parts) was added to poly(NVP-co-DMAEMA) or PVPDM copolymer (5 parts) from Example 15 in dimethylformamide (10 parts). After the reaction was run at 75-80° C. for 6 hours, the copolymer (PVPDM-g-CPD) was precipitated with ether. Then the resultant polymer in dimethylformamide (10 parts) was directly used to react with heptanal (HAL, 2 parts), in the presence of p-toluenesulfonic acid (0.3 part) at room temperature overnight. After precipitation and washing with ether, the polymer (PVPDM-g-CPDHAL) was dried and stored in a vacuum oven.

### Example 38

Synthesis of CALC-containing poly(NVP-co-DMAEMA) copolymer: CPD (1.3 parts) was added to poly(NVP-co-DMAEMA) or PVPDM copolymer (5 parts) from Example 15 in dimethylformamide (10 parts). After the reaction was run at 75-80° C. for 6 hours, the copolymer (PVPDM-g-CPD) was precipitated with ether. Then the resultant polymer in dimethylformamide (10 parts) was directly used to react with citronellal (CAL, 3 parts), in the presence of p-toluenesulfonic acid (0.3 part) at room temperature overnight. After precipitation and washing with ether, the polymer (PVPDM-g-CPDCAL) was dried and stored in a vacuum oven.

### Example 39

Synthesis of ATPC-containing poly(NVP-co-DMAEMA) copolymer: CPD (1.3 parts) was added to poly(NVP-co-DMAEMA) or PVPDM copolymer (5 parts) from Example 15 in dimethylformamide (10 parts). After the reaction was run at 75-80° C. for 6 hours, the copolymer (PVPDM-g-CPD) was precipitated with ether. Then the resultant polymer in dimethylformamide (10 parts) was directly used to react with acetophenone (ATP, 2.5 parts), in the presence of p-toluenesulfonic acid (0.3 part) at room temperature overnight. After precipitation and washing with ether, the polymer (PVPDM-g-CPDATP) was dried and stored in a vacuum oven.

### Examples 40-49

### FT-IR Characterization

### Example 40

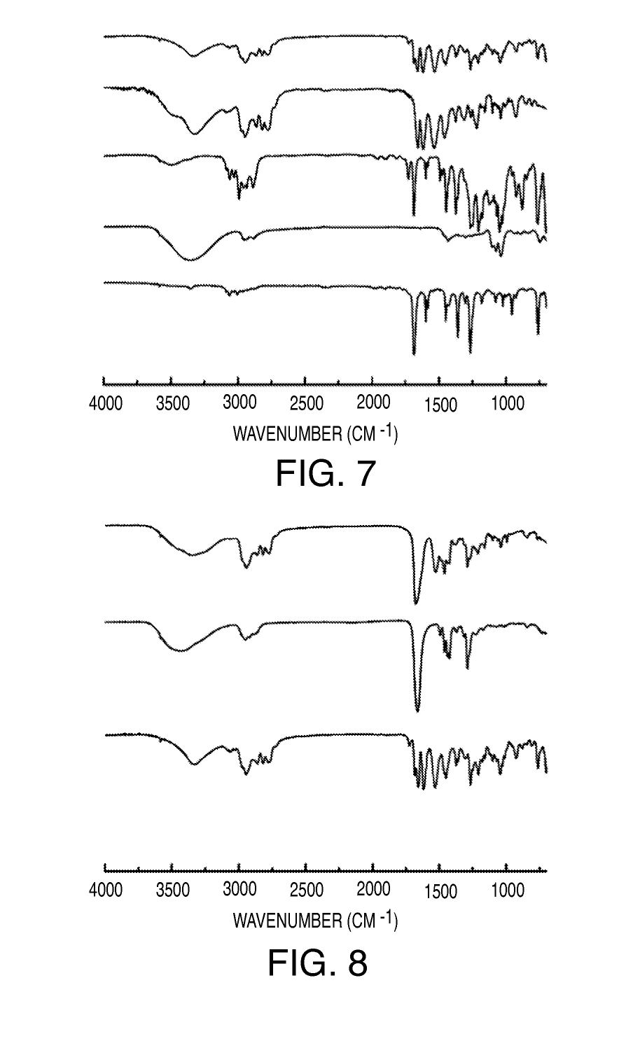

FIG. 7 shows the Fourier transform-infrared (FT-IR) spectra of ATP, CPD, AC, DMAPMAm, and DAC. Regarding ATP, CPD, and AC, the disappearance of a strong peak at 3349 cm⁻¹ (hydroxyl group from CPD) and the appearance of the peaks at 1069, 1047, 924, and 879 (acetal group) on AC confirmed completion of the cyclic acetal fragrance intermediate formation. By comparing AC, DMAPMAm, and DAC, the formation of the peak at 3333 cm⁻¹ for quaternary ammonium chloride; the peaks at 1724, 1686, 1099, 1044 and 924 from AC; and the peaks at 1658 (amide I), 1619 (C═C), and 1531 (amide II) from DMAPMAm confirmed the formation of DAC, which contains acetal ((R₁)₂C(O—CR)2), carbon-carbon double bond (C═C), amide (I and II), and quaternary ammonium chloride functionalities.

### Example 41

FIG. 8 shows the FT-IR spectra of DAC, PVP, and poly(NVP-co-DAC). The formation of a broad peak at 3362 cm⁻¹, which covers quaternary ammonium chloride from DAC and substituted amide from NVP (3434); peaks at 2946, 2860, 2821, and 2777 (multi —CH₂— from both DAC and NVP); a peak at 1676 that covers both carbonyl groups of NVP (amide, 1664) and DAC (amide I, 1686); a peak at 1530 (amide II from DAC); and peaks at 1099, 1045, 925, and 844 (acetal from DAC) on poly(NVP-co-DAC) confirmed that the purified polymer contains acetal, NVP, and quaternary ammonium chloride functionalities.

### Example 42

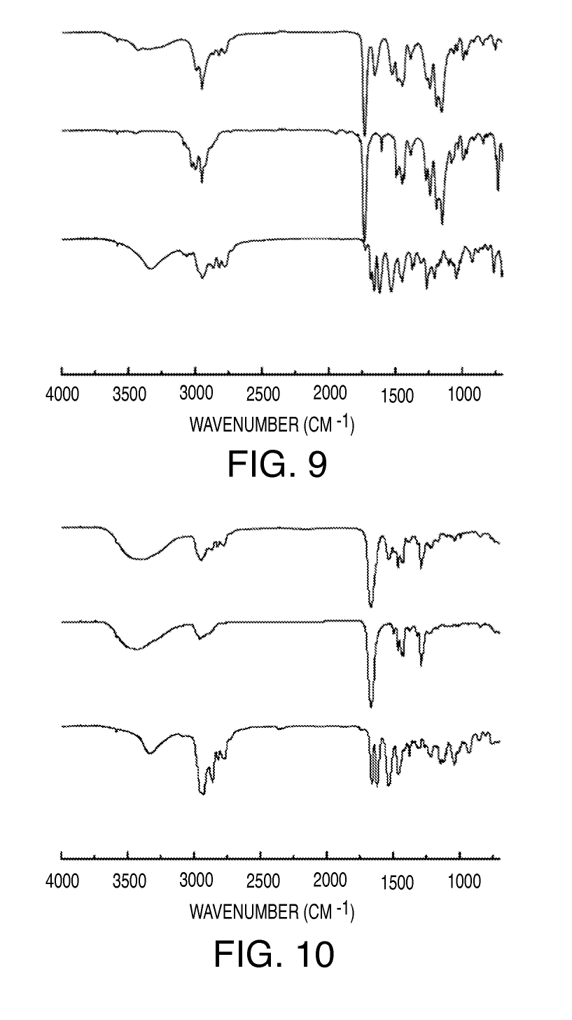

FIG. 9 shows the FT-IR spectra of DAC, PMMA, and poly(MMA-co-DAC). The formation of a broad peak at 3350 cm⁻¹, which represents quaternary ammonium chloride from DAC; peaks at 2989, 2948, 2869, 2822, and 2777 (multi —CH₂— from both DAC and MMA); the peak at 1730 (carbonyl group from MMA ester); the peaks at 1655 (amide I) and 1525 (amide II) from DAC; and peaks at 1156, 1038, 946, and 843 (acetal from DAC) on poly(MMA-co-DAC) confirmed that the purified polymer contains acetal, MMA, and quaternary ammonium chloride functionalities.

### Example 43

FIG. 10 shows the FT-IR spectra of DHC, PVP, and poly(NVP-co-DHC). The formation of a broad peak at 3400 cm⁻¹, which covers quaternary ammonium chloride from DHC (3327) and substituted amide from NVP (3434); peaks at 2947, 2868, 2822, and 2774 (multi —CH₂— from both DHC and NVP); the peak at 1664 that covers both carbonyl groups of NVP (amide, 1664) and DHC (amide I, 1658); the peak at 1529 (amide II); and peaks at 1100, 1038, 994, and 845 (acetal from DHC) on poly(NVP-co-DHC) confirmed that the purified polymer contains acetal, NVP, and quaternary ammonium chloride functionalities.

### Example 44

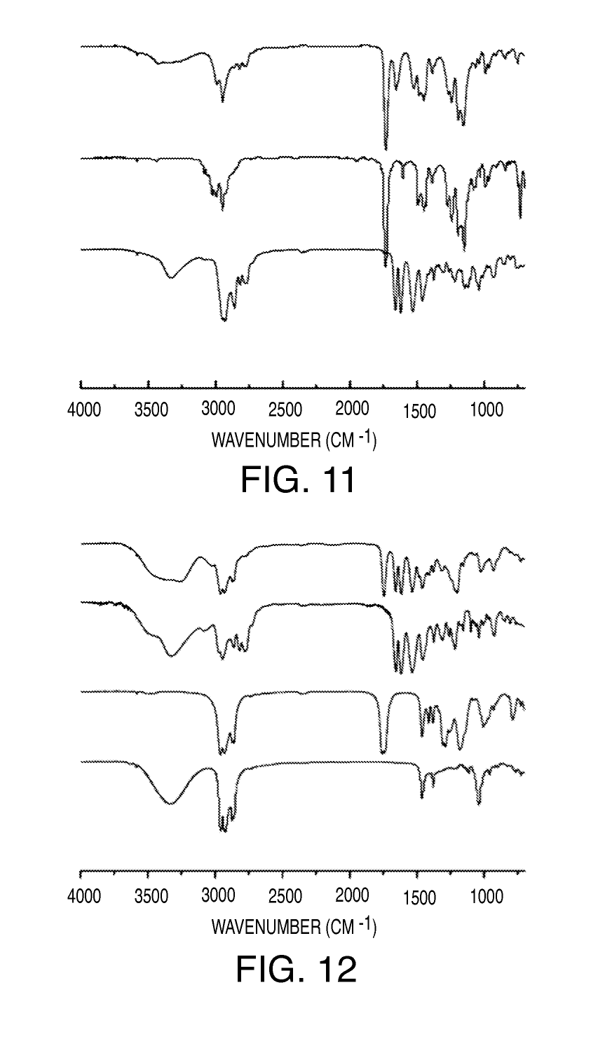

FIG. 11 shows the FT-IR spectra of DHC, PMMA, and poly(MMA-co-DHC). The formation of a broad peak at 3419 cm⁻¹, which represents quaternary ammonium chloride; peaks at 2990, 2948, 2868, 2823, and 2782 (multi —CH₂— from both DHC and MMA); the peak at 1729 (carbonyl group from MMA ester); peaks at 1642 (amide I) and 1528 (amide II) from DHC; and peaks at 1157, 1033, 991, and 845 (acetal from DHC) on poly(MMA-co-DHC) confirmed that the purified polymer contains acetal, MMA, and quaternary ammonium chloride functionalities.

### Example 45

FIG. 12 shows the FT-IR spectra of EH, ECA, DMAPMAm, and DECA. Regarding EH and ECA, the disappearance of a broad peak at 3337 cm⁻¹ (hydroxyl group from EH) and the appearance of peaks at 1760 and 1755 (ester group) on ECA confirmed completion of the reaction between EH and CAA. By comparing ECA, DMAPMAm, and DECA, the formation of the broad peaks at 3230-3600 cm⁻¹, which cover both quaternary ammonium chloride and amide NH-from DMAPMam; peaks at 2994, 2959, 2931, 2873, and 2863 (multi —CH₂— from both ECA and DMAPMAm); the peak at 1658 (amide I from DMAPMAm); the peak at 1614 cm⁻¹ for C═C from DMAPMAm; and the peak at 1537 (amide II, DMAPMAm) confirmed the formation of DECA, which contains ester, carbon-carbon double bond, quaternary ammonium chloride, and amide functionalities.

### Example 46

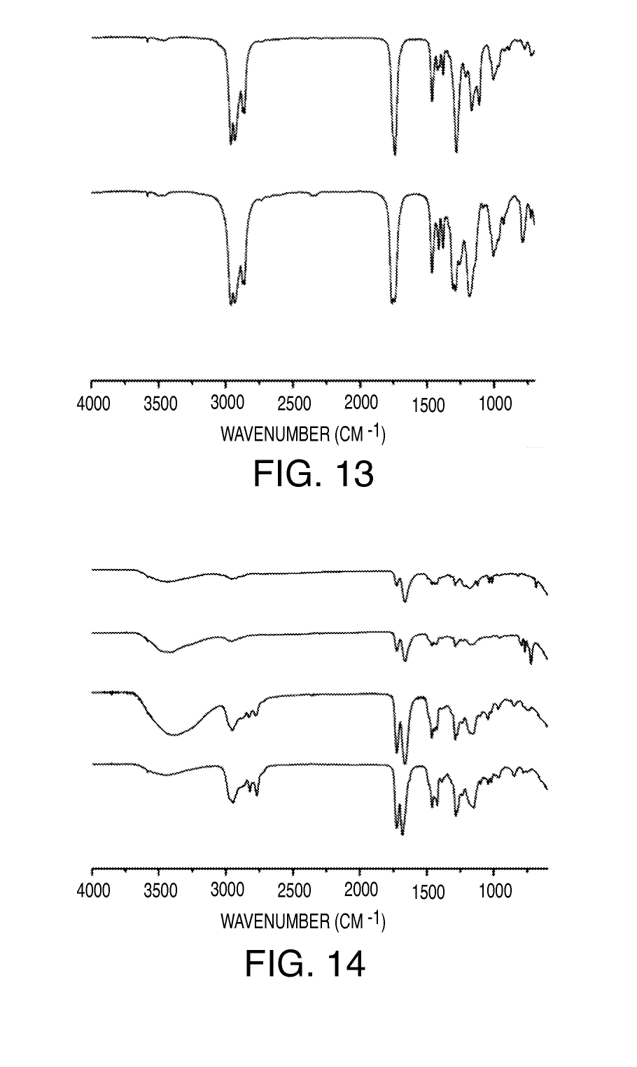

FIG. 13 shows the FT-IR spectra of ECA and EBA. For both spectra, the disappearance of peaks at 3200-3600 cm⁻¹ (hydroxyl group from EH) and the appearance of the new peaks at 1760 and 1755 (ester group for ECA) and 1739 (ester group for EBA) confirmed the completion of the reaction between EH and CAA and/or between EH and BAA.

### Example 47

FIG. 14 shows the FT-IR spectra of poly(NVP-co-DMAEMA) or PVPDM (spectrum at the bottom), PVPDM-g-CPD, PVPDM-g-CPDAld, and PVPDM-g-CPDAIdEH (spectrum at the top). The shift of a peak from 3441 to 3391 and formation of peaks at 1446 and 739 on PVPDM-g-CPD confirmed the quaternary ammonium chloride formation between CPD and DMAEMA. The appearance of the peaks at 955, 766, and 722 on PVPDM-g-CPDAId confirmed the formation of aldehyde groups as compared to those shown on PVPDM-g-CPD. The reduction of the peak at 1723 (ester), which represents the ester-linkages for NVP, DMAEMA, and aldehyde; the disappearance of peaks at 955, 790, 766, and 722; and the formation of peaks at 1221, 1034, 1010, 820, and 682 on PVPDM-g-CPDAIdEH confirmed both the formation of the acetal linkage between the aldehyde group on PVPDM-g-CPDAId and the hydroxyl group on EH and the disappearance of an aldehyde group on PVPDM-g-CPDAIdEH.

### Example 48

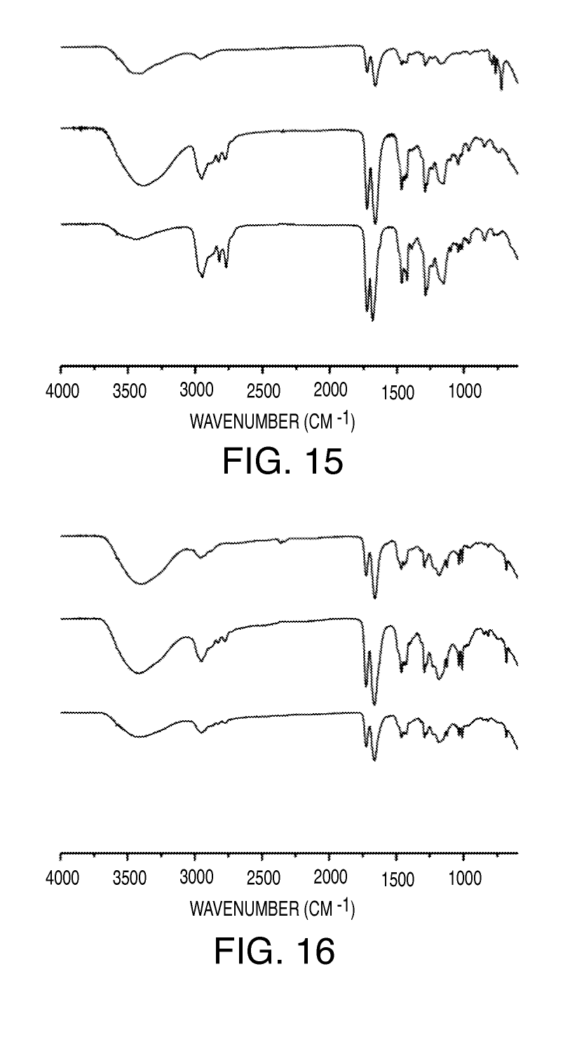

FIG. 15 shows the FT-IR spectra of PVPDM (bottom), PVPDM-g-CPD, and PVPDM-g-CPDHAL (top). The explanation for PVPDM and PVPDM-g-CPD is the same as that for FIG. 13. Regarding PVPDM-g-CPDHAL, the appearance of peaks at 1122, 1034, 1011, and 682 confirmed the formation of the acyclic acetal linkage between the 1,2-diol group on PVPDM-g-CPD and the aldehyde group on HAL.

### Example 49

FIG. 16 shows the FT-IR spectra of PVPDM-g-CPDHAL (bottom), PVPDM-g-CPDCAL, and PVPDM-g-CPDATP. HAL and CAL are an aldehyde-based fragrance whereas ATP is a ketone-based fragrance. Comparing all three spectra demonstrates that all three polymers showed peaks at 1122, 1034, 1011, and 682, which confirmed the formation of the acyclic acetal linkage between the 1,2-diol group on PVPDM-g-CPD and the aldehyde group on both HAL and CAL or ketone group on ATP.

The triggerable composition of the disclosure can be applied to a substrate such as an absorbent article, or layer within an absorbent article, by any number of known applications or printing techniques. For example, the triggerable composition of the present disclosure can be deposited on a substrate by various surface deposition or printing methods such as brushing, flexographic printing, gravure roll printing , stamping, screen print, spraying techniques, dip and squeeze, and digital print methods. Further, the composition can be applied in a melt form and allowed to solidify on a treated substrate. As also noted, the composition can be part of a lotion, cream, or medicament as well.

Placement of the triggerable composition can be on any number of substrates. The substrate sheets can for instance, include nonwoven or woven sheets. Such sheets can include synthetic or natural fibrous materials such as for example, extruded spunbond, and meltblown webs, bonded carded webs, or airlaid materials, spun cellulosic, wool or synthetic yarns. Such sheets can further include cellulosic-based dry or wet laid tissue or paper sheets. Additionally, such substrates can include film or foam sheets, laminates of film, foam and fibrous layers, or laminates of multiple fibrous, film and foam layers. Such substrates/sheets can be placed as layers within medical or beauty care articles, personal care hygienic articles such as absorbent articles, or can themselves serve as the absorbent article, such as as a towel, tissue or wipe. Further, such triggerable composition can be used as components in lotions, creams, and medicaments, such as tablets or suppositories.

Placement of such composition in an article/absorbent article can be across the entire article's longitudinal and transverse or lateral (width) dimensions, or layer of an article, or alternatively, can be limited to certain locations within the article, or layer(s) on the article. For example, such composition can be placed at a location specifically designed to contact aqueous—based waste, such as a highly probable “soiling area” in an article's or layer's central crotch region. Such treated layers can include the topsheet layer, backsheet layer (inner surface), or absorbent core layer. Other interior-positioned layers can also be treated with the coating composition. In an alternative aspect, it can be desirable to limit the placement of the coating formulation to certain locations on an absorbent article that would not directly impact the absorbency pathways of an article, such as on an inside surface of a backsheet layer (as opposed to a topsheet layer or absorbent core layer), or side areas of a topsheet layer, absorbent core layer, or other interior situated layer.

In a first particular aspect, a triggerable composition for one-stage, controlled release of a functional chemical includes a functional monomer having a structure selected from the group consisting of

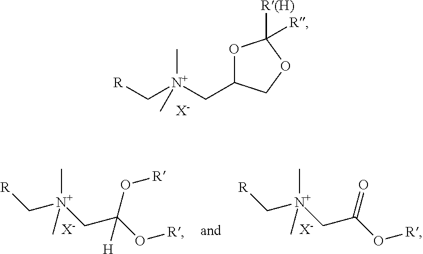

wherein R is a polymerizable portion, N⁺X⁻ is a quaternary ammonium halide, and R′ and R″ are hydrocarbon-containing groups, wherein at least one of R′ and R″ includes a fragrance.

A second particular aspect includes the first particular aspect, wherein at least one of R′ and R″ is a ketone.

A third particular aspect includes the first and/or second aspect, wherein R″ is an aldehyde.

A fourth particular aspect includes one or more of aspects 1-3, wherein R′ is an alcohol.

A fifth particular aspect includes one or more of aspects 1-4, further comprising a polymer including the functional monomer.

A sixth particular aspect includes one or more of aspects 1-5, further comprising a copolymer including the functional monomer.

A seventh particular aspect includes one or more of aspects 1-6, wherein the quaternary ammonium halide is selected from the group consisting of bromide, chloride, iodide, and combinations thereof.

An eighth particular aspect includes one or more of aspects 1-7, wherein the quaternary ammonium halide is an unstable trigger for a cyclic acetal-, acyclic acetal-, or ester-linked fragrance in the presence of water.

A ninth particular aspect includes one or more of aspects 1-8, further comprising a crosslinked polymer network containing the functional monomer.

A tenth particular aspect includes one or more of aspects 1-9, wherein the crosslinked polymer network includes a hydrogel, semi-IPN, full-IPN, or polymer blend.

An eleventh particular aspect includes one or more of aspects 1-10, further comprising a copolymer of a hydrophilic comonomer and the functional monomer.

A twelfth particular aspect includes one or more of aspects 1-11, further comprising a copolymer of a hydrophobic comonomer and the functional monomer.

A thirteenth particular aspect includes one or more of aspects 1-12, further comprising a copolymers of an amphiphilic comonomer and the functional monomer.

In a fourteenth particular aspect, a coating includes a triggerable composition for one-stage, controlled release of a functional chemical, the composition including a functional monomer having a structure selected from the group consisting of

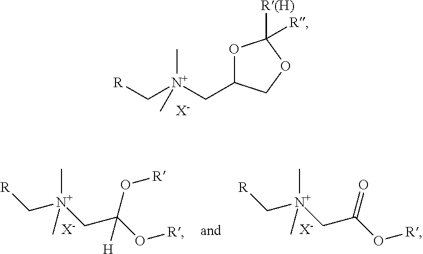

wherein R is a polymerizable portion, N+X− is a quaternary ammonium halide, and R′ and R″ are hydrocarbon-containing groups, wherein at least one of R′ and R″ includes a fragrance.

In a fifteenth particular aspect, a skin care element including a triggerable composition for one-stage, controlled release of a functional chemical, the composition including a functional monomer having a structure selected from the group consisting of

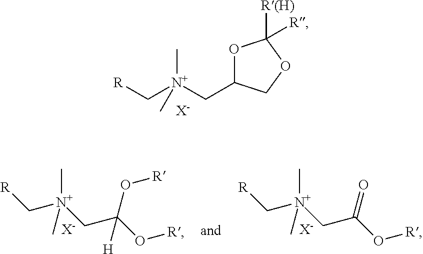

wherein R is a polymerizable portion, N⁺X⁻ is a quaternary ammonium halide, and R′ and R″ are hydrocarbon-containing groups, wherein at least one of R′ and R″ includes a skin active chemical

A sixteenth particular aspect includes the fifteenth particular aspect, wherein at least one of R′ and R″ is a ketone.

A seventeenth particular aspect includes the fifteenth and/or the sixteenth aspects, wherein R″ is an aldehyde.

An eighteenth particular aspect includes one or more of aspects 15-17, wherein R′ is an alcohol.

A nineteenth particular aspect includes one or more of aspects 15-18, further comprising a polymer including the functional monomer.

A twentieth particular aspect includes one or more of aspects 15-19, wherein the skin active chemical is an anitoxidant.

The present disclosure has been described in general and in detail by means of examples. Persons of skill in the art understand that the disclosure is not limited necessarily to the aspects specifically disclosed, but that modifications and variations can be made without departing from the scope of the disclosure as defined by the following claims or their equivalents.

We claim:

1. A triggerable composition for one-stage, controlled release of a functional chemical comprising:
a functional monomer having a structure selected from the group consisting of 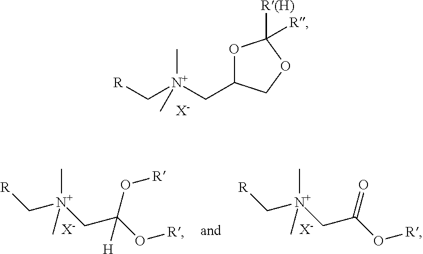
wherein R is a polymerizable portion, N⁺X⁻ is a quaternary ammonium halide, and R′ and R″ are hydrocarbon-containing groups, wherein at least one of R′ and R″ includes a fragrance.

2. The triggerable composition of claim 1, wherein at least one of R′ and R″ is a ketone.

3. The triggerable composition of claim 1, wherein R″ is an aldehyde.

4. The triggerable composition of claim 1, wherein R′ is an alcohol.

5. The triggerable composition of claim 1, further comprising a polymer including the functional monomer.

6. The triggerable composition of claim 1, further comprising a copolymer including the functional monomer.

7. The triggerable composition of claim 1, wherein the quaternary ammonium halide is selected from the group consisting of bromide, chloride, iodide, and combinations thereof.

8. The triggerable composition of claim 1, wherein the quaternary ammonium halide is an unstable trigger for a cyclic acetal-, acyclic acetal-, or ester-linked fragrance in the presence of water.

9. The triggerable composition of claim 1, further comprising a crosslinked polymer network containing the functional monomer.

10. The triggerable composition of claim 9, wherein the crosslinked polymer network includes a hydrogel, semi-IPN, full-IPN, or polymer blend.

11. The triggerable composition of claim 1, further comprising a copolymer of a hydrophilic comonomer and the functional monomer.

12. The triggerable composition of claim 1, further comprising a copolymer of a hydrophobic comonomer and the functional monomer.

13. The triggerable composition of claim 1, further comprising a copolymers of an amphiphilic comonomer and the functional monomer.

14. A coating comprising:
a triggerable composition for one-stage, controlled release of a functional chemical, the composition including a functional monomer having a structure selected from the group consisting of 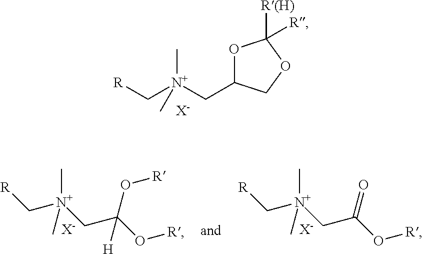
wherein R is a polymerizable portion, N+X− is a quaternary ammonium halide, and R′ and R″ are hydrocarbon-containing groups, wherein at least one of R′ and R″ includes a fragrance.

15. A skin care element comprising:
a triggerable composition for one-stage, controlled release of a functional chemical, the composition including a functional monomer having a structure selected from the group consisting of 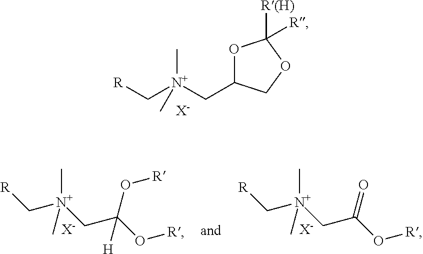
wherein R is a polymerizable portion, N⁺X⁻ is a quaternary ammonium halide, and R′ and R″ are hydrocarbon-containing groups, wherein at least one of R′ and R″ includes a skin active chemical.

16. The skin care element of claim 15, wherein at least one of R′ and R″ is a ketone.

17. The skin care element of claim 15, wherein R″ is an aldehyde.

18. The skin care element of claim 15, wherein R′ is an alcohol.

19. The skin care element of claim 15, further comprising a polymer including the functional monomer.

20. The skin care element of claim 15, wherein the skin active chemical is an anitoxidant.
